(19) United States
(12) Patent Application Publication
(10) Pub. No.: US 2014/0171758 A1
(43) Pub. Date: Jun. 19, 2014

(54) Integrated Portable Medical Diagnostic System

(71) Applicant: Stephen Teni Ayanruoh, Yonkers, NY (US)

(72) Inventor: Stephen Teni Ayanruoh, Yonkers, NY (US)

(21) Appl. No.: 14/233,755

(22) PCT Filed: Jul. 18, 2012
(86) PCT No.: PCT/US12/47243

§ 371 Date: Jan. 19, 2014

Related U.S. Application Data
(60) Provisional application No. 61/572,643, filed on Jul. 20, 2011.

(51) Int. Cl. A61B 5/0205; A61B 5/00

(52) U.S. Cl. CPC A61B 5/02055; A61B 5/68
USPC 600/301

(57) ABSTRACT

A portable integrated self-contained rechargeable (100) with AC-DC direct current battery system mounted within an instrument case 99 for housing a foldable keyboard (119), a display (112) and plurality of medical test and treatment modules. It optionally includes a suitable DC to AC inverter to supply AC power. The system includes at least one USB port (154) for adding external test and treatment modules on ad hoc basis. It also includes full duplex data and information input, processing (216), output and communications interface to and from sensors, transducers, networks and other data/information generation or consumption modules, devices and subsystems. It is also known by its three trademark names “Hospital-in-a-Box”, RK Ruskat (Stylized) and SCOMET.

### RELATED PRIORITY DOCUMENT

This application is related to U.S. Provisional application No. 61/572,643 filed on Jul. 20, 2011 entitled, “Integrated Portable Medical Diagnostic System” by the same inventor and applicant Dr. Stephen Teni Ayanruoh who claims priority therefrom.

### BACKGROUND

This invention relates generally to methods, devices and system for patient diagnostics and tests for use by medical personnel when making house calls or emergency ambulance service or any triage site of temporary and/or emergency medical services in case of natural or manmade catastrophe, especially in remote and rural areas where the Doctor Patent ratio is greater than 1:100,000. It comprises a plurality of hardware and software modules in a suitable self-contained integrated suitcase complete with its own power supply, communications interface and a personal computer or microprocessor or equivalent electronic discreet logic circuits. It is also known by its three trademark names “Hospital-in-a-Box”, RKRuskat (Stylized) and Scomet.

### THE PROBLEM

The problem with prior art diagnostic systems is that they are not suitable for house calls and concomitant diagnostic testing and data collection, processing and recording and communications with main frames of the hospital or primary computer of the physicians. The prior art devices are also neither modular nor user friendly nor flexible enough to permit various permutations and combinations of test at will and on demand that a patient may require or the exigencies of the circumstances may dictate.

### THE SUMMARY

The inventor's motivation for this invention is grounded in his healthcare philosophy in his own words, “It is the right of every citizen of the world irrespective of their color, race, political affiliations, religion, sexual orientations or sex should have access to basic comprehensive health care. I am also trying to increase the productivity and efficiency of doctors in the developing world with Hospital-In-a-Box because the ratio of their doctors to their population is about 3:200,000” The invention is an improvement over stationary medical diagnostic systems. It comprises methods, devices and system for patient diagnostics and tests for use by medical personnel when making house calls or emergency ambulance service especially at triage or other natural or manmade catastrophic sites for saving lives and restoring injured or otherwise inflicted patients to good health. It comprises a plurality of hardware and software modules in a suitable self-contained suitcase complete with its own power supply, communications interface and a personal computer or microprocessor or equivalent electronic discreet logic circuits. It is also known by its three trademark names “Hospital-in-a-Box”, RKRuskat (Stylized) and Scomet.

### PRIOR ART

A formal preliminary prior art patentability and novelty search was neither conducted nor commissioned, but the inventor is not only a credentialed Doctor of Medicine but also at the cutting edge of this technology and therefore is intimately familiar with the prior art. No prior art exists to match the functionality and user friendliness of the present invention. Accordingly none of the prior art devices singly or even in combination provides all of the features and objectives established by the inventor for this system as enumerated below.

The best that can be obtained in prior art is partial solutions which are complicated, cumbersome, burdensome, expensive and impractical. This application at bar is the most cost effective solution to the problem since the physicians stopped making house calls.

### OBJECTIVES

1. It is an objective of this invention to provide methods, devices and system for an integrated portable medical diagnostic system to serve as a Hospital-in-a-Box.
2. Another objective of this invention is that it be particularly suitable for providing emergency competent and professional medical services at triage or other emergency man made or natural disaster sites.
3. Another object of this invention is that it be capable of handling mass casualties, large number of patients efficiently even in rural or otherwise hard to reach areas.
4. Another objective of this invention is that all sensors and transducers, modules and subsystems be plug compatible.
5. Another objective of this invention is to compensate for the ageing of the components and sub-systems of the system.
6. Another objective of this invention is that the functionality of the system is not compromised in any manner including ergonomics, user-friendliness, and result for each mode of operation.
7. Another objective of this invention is having all desired functions embedded and be intuitive as well as clearly visible when enabled.
8. Another objective of this invention is that it be energy efficient.
9. Another objective of this invention is that it is easy to use even intuitive that requires little additional training if any.
10. Another objective of this invention is that it be environmentally friendly to prevent spread of disease(s) being cured.
11. Another objective of this invention is that it be made of modules standard parts easily interface-able to each other.
12. Another objective of this invention is that it is capable of accommodating new modules as needs change and new technologies become available for curing new disease(s) or afflictions.
13. Another objective of this invention is that new modules can be added via a USB (Universal Serial Bus) or other portal of duplex medical data and information exchange.
14. Another objective of this invention is that the database can be shared with a plurality of devices and networks of different types with no or little additional simple interface(s).
15. Another objective of this invention is that it meets all federal, state, local and other private standards, guidelines and recommendations with respect to safety, environment, and quality and energy consumption.
16. Another objective of this invention is that it be elegantly simple in concept and design.
17. Another objective of this invention is that it be suitable for use with all types of patients regardless of the handicap of the patient.
18. Another objective of this invention is that it be easy to assemble, service, transport and store.
19. Another objective of this invention is that it be useable in all types of locations including ambulance, home, schools, work, office, shop floor, factory floor triage and other emergency sites in times of need at short notice even in remote and hard to reach venues, nations, continents and space colonies etc.
20. Another objective of this invention is that it be user friendly and patient transparent.
21. Another objective of this invention is that it lends itself to multiple uses.
22. Another objective of this invention is that it be reliable such that it practically never fails and requires little or no maintenance.
23. Another objective of this invention is that it be made from biodegrade materials to the extent practical and cost effective.
24. Another objective of this invention is that it be long lasting with high MTBF (Mean Time Between Failures) made from durable material,
25. Another objective of this invention is that it have small foot print
26. Another objective of this invention is that it have lower life cycle cost.
27. Another objective of this invention is numerous medical instrument modules are packaged and integrated into a single portable carrying case which is easy to roll as well as easy to carry on non-roll able surfaces to reposition at or near patent/doctor table(s) and platforms.
28. Another objective of this invention is that in conjunction with a knapsack it can be used for military and remote locations.
29. Another objective of this invention is that it provide power options such as direct current batter power, even automobile 12 volt direct current, building alternating current power and generator power of desired voltages and currents and hence the power including means for converting one form of power to another including rectification of power at the power source and/or at the point of consumption or at the Hospital-in-a-Box itself.
30. Another objective of this system is Satellite modem to transfer test data wireless to central computer/EMS service.
31. Another objective is to provide a Video camera scope that includes an LCD display mounted to it for ease use.
32. Yet another camera related objective is to provide a scope that is capable of real time video streaming & single frame capture of digital images.
33. Another objective of this invention Video camera scope is to incorporate an LED brightness control means on handle.
34. Another objective of this invention is that the video camera scope includes a focus adjustment means on handle or other portable device.
35. Another objective of this system is that it includes the flexibility to mount special attachment to slightly pressurize the eardrum through the specula via a hand bulb.
36. Another objective of this invention is that the pictures can be taken for future reference that will stay in the patient's folder for the life of the patient as permitted by the applicable privacy laws.
37. Another objective of this invention that the documented pictures can make it easier for the physician to determine if the patient's condition is improving satisfactorily or getting worse.
38. Another objective e of this invention is to provide immediate onsite real time feedback to the patient, such that the patient can view concurrently in parallel exactly what the physician is seeing on the computer screen at the same time, allowing for the patients to better understand their condition.
39. Another objective of this invention is to make it once again practical for physicians and other medical professionals to make house calls conveniently, affordably and cost effectively.
40. Another objective of this invention is to encourage Doctors to serve rural areas by providing them with means and modes for service there.
41. Another objective of this invention is to make American medical practice once again respectable and competitive on global basis.
42. Other objectives of this invention reside in its simplicity, elegance of design, ease of manufacture, service and use and even aesthetics as will become apparent from the following brief description of the drawings and the detailed description of the concept embodiment.

### BRIEF DESCRIPTION OF THE DRAWINGS

Following is a brief description of the several views of the drawings complete with reference numerals wherein last two least significant digits represent the item (not necessarily tangible) number and the left most one or two digits represent the figure number.

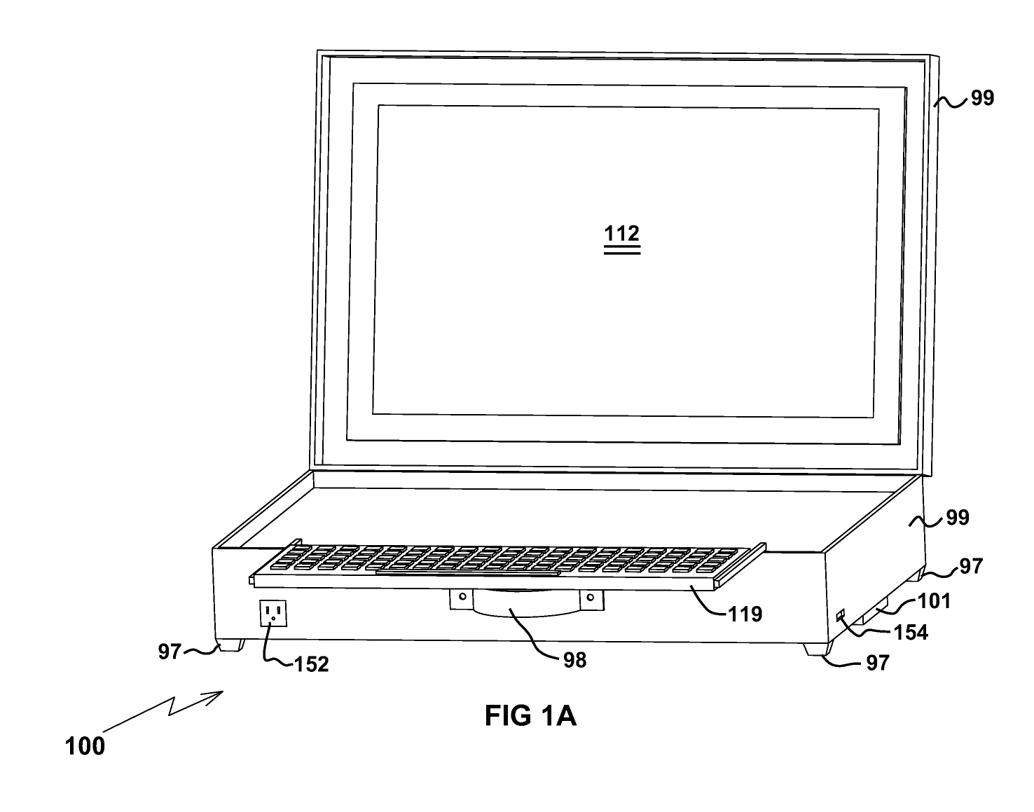

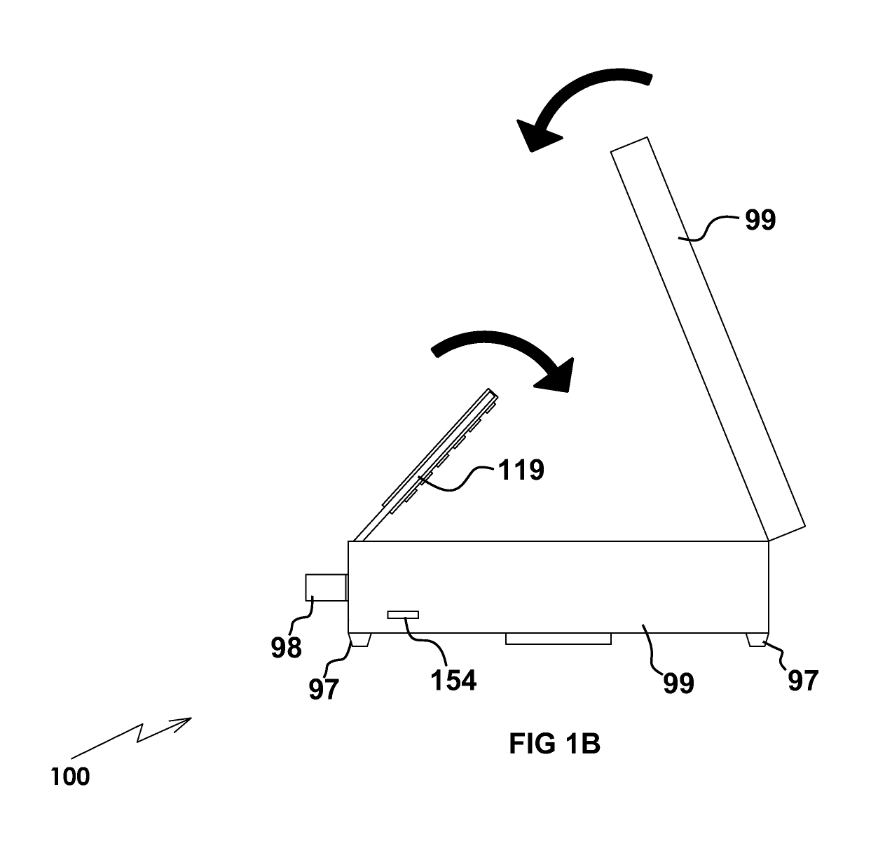

a) FIG. 1A is a 3D perspective view of the Integrated Portable Medical Diagnostic System.

b) FIG. 1B is a right side view of the Hospital-in-a-Box in partially open/closed position.

c) FIG. 1C is side view of the invention in fully closed ready to roll position with retractable handle extended.

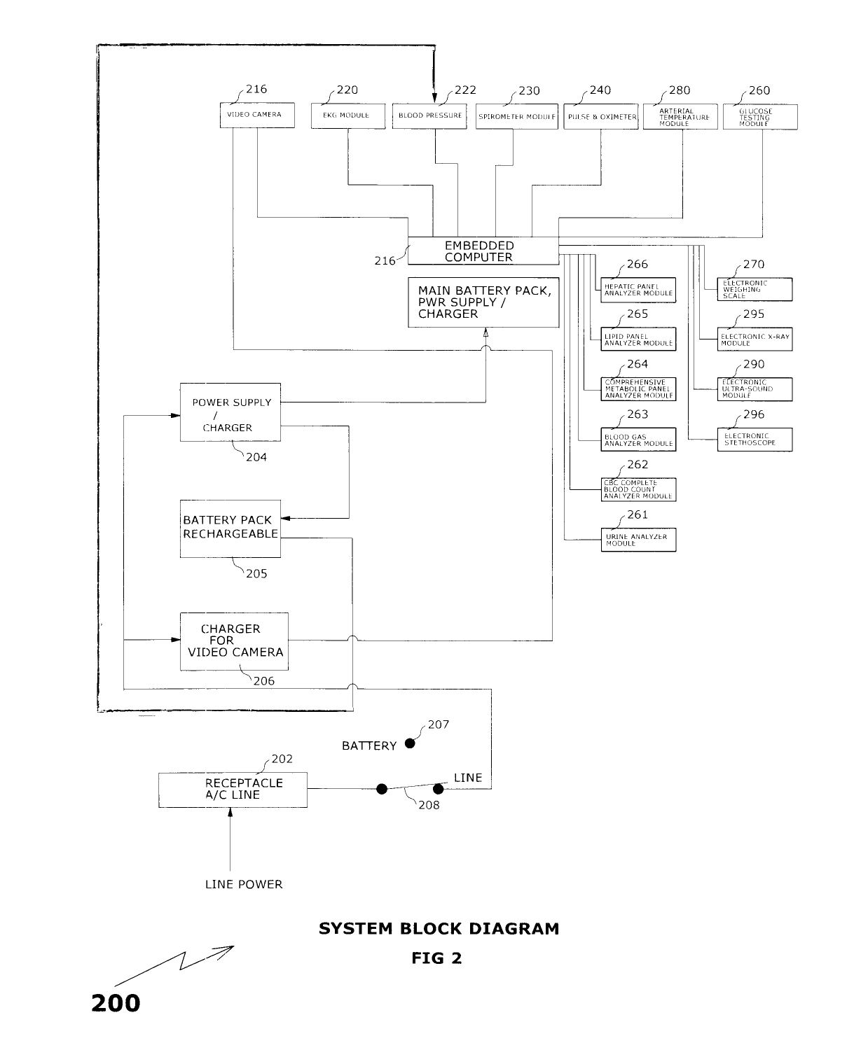

d) FIG. 2 is a system modules block diagram.

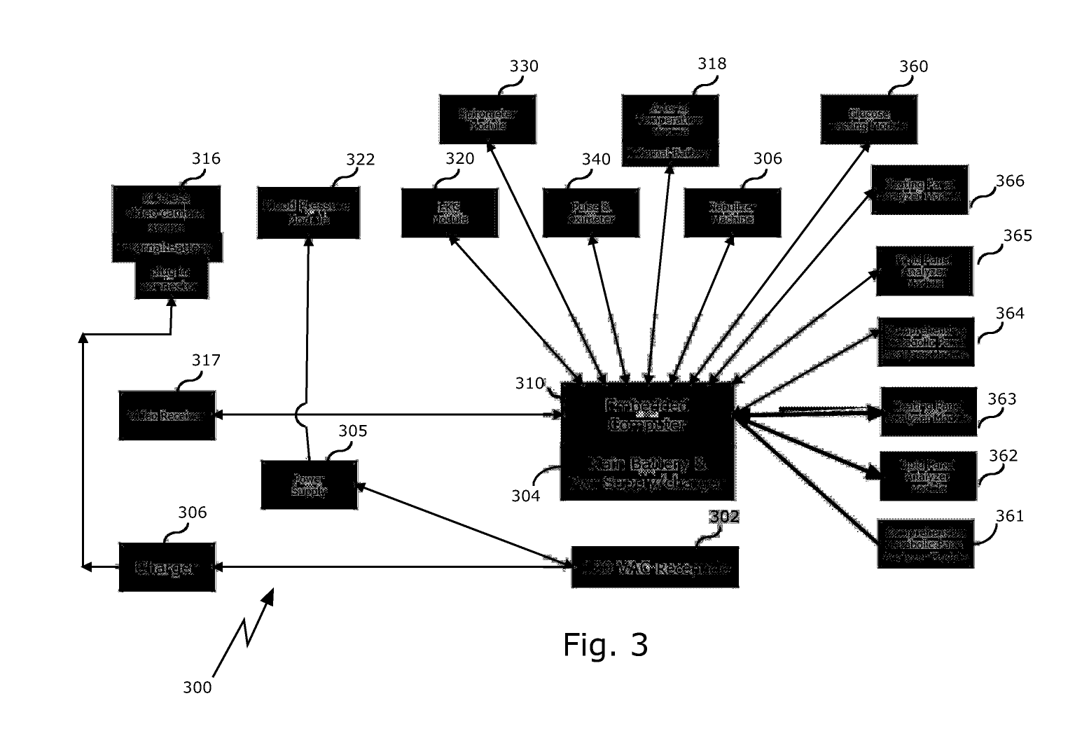

e) FIG. 3 is another somewhat more detailed rendition of the system modules block diagram of FIG. 2.

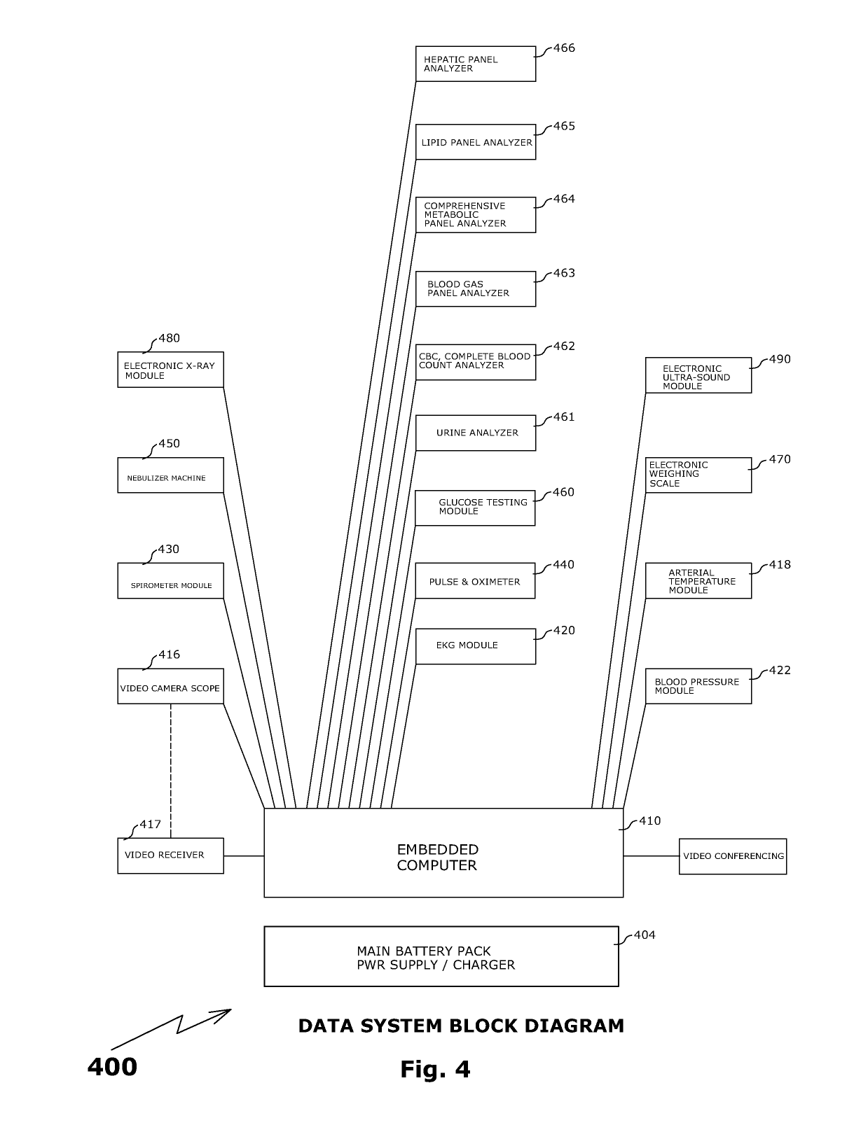

f) FIG. 4 is depicts data system block diagram.

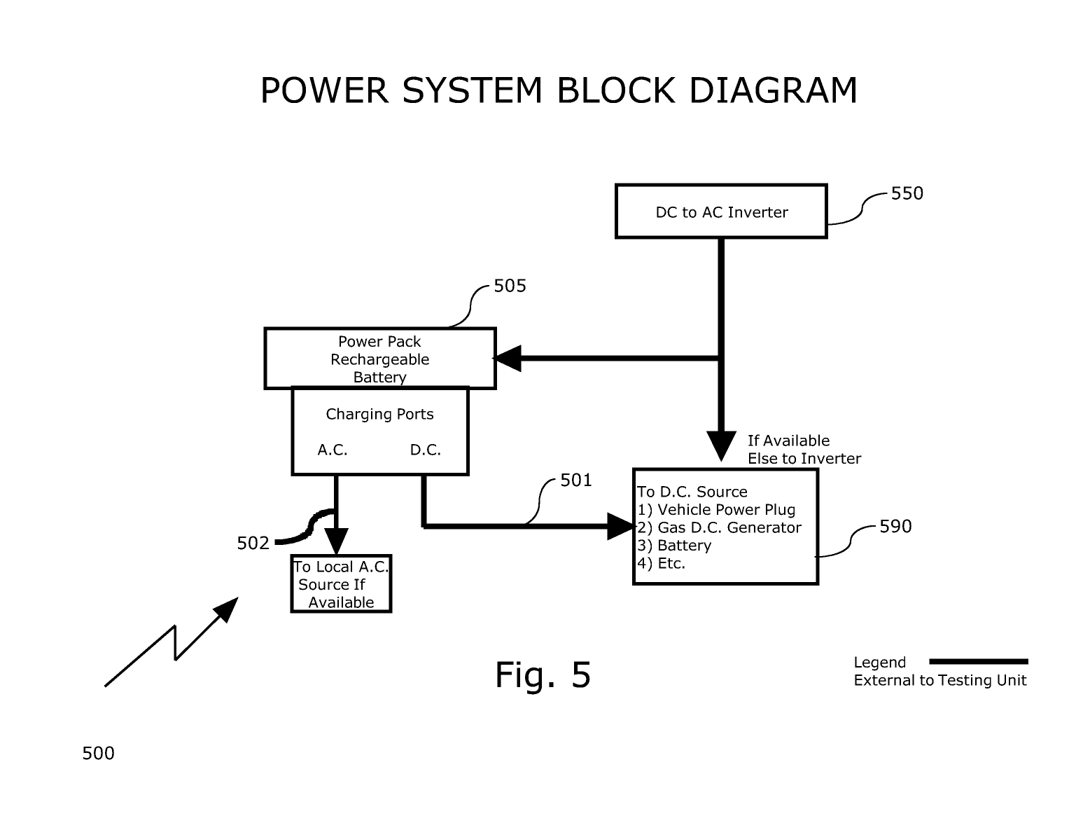

g) FIG. 5 shows power sub-system block diagram

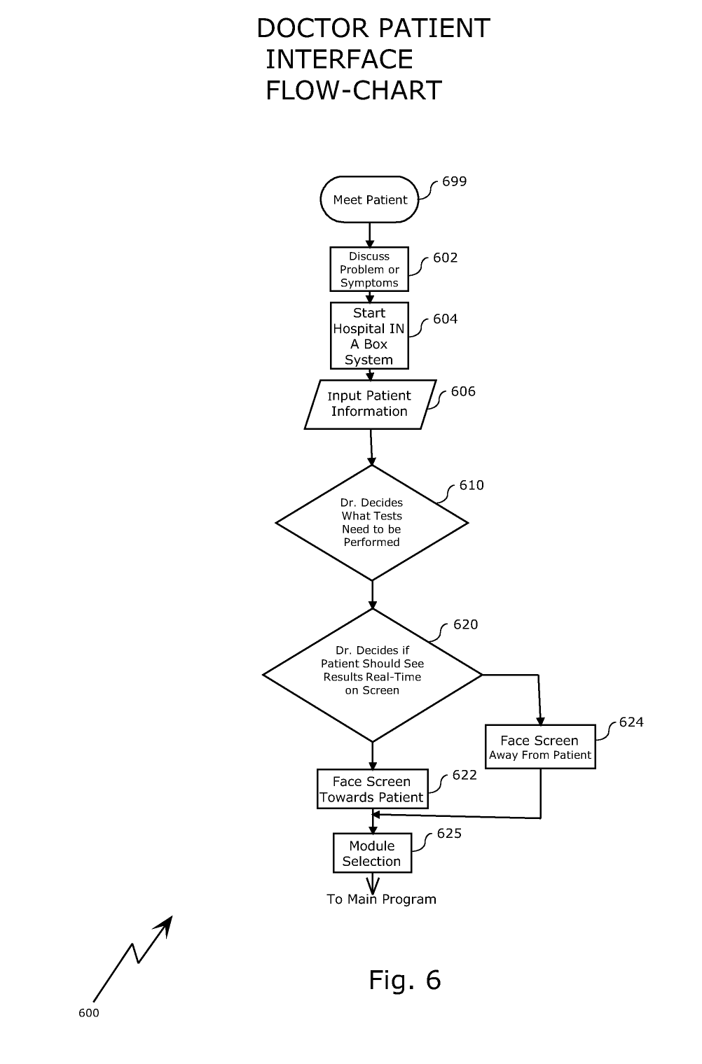

h) FIG. 6 is a flow chart of doctor patient interface.

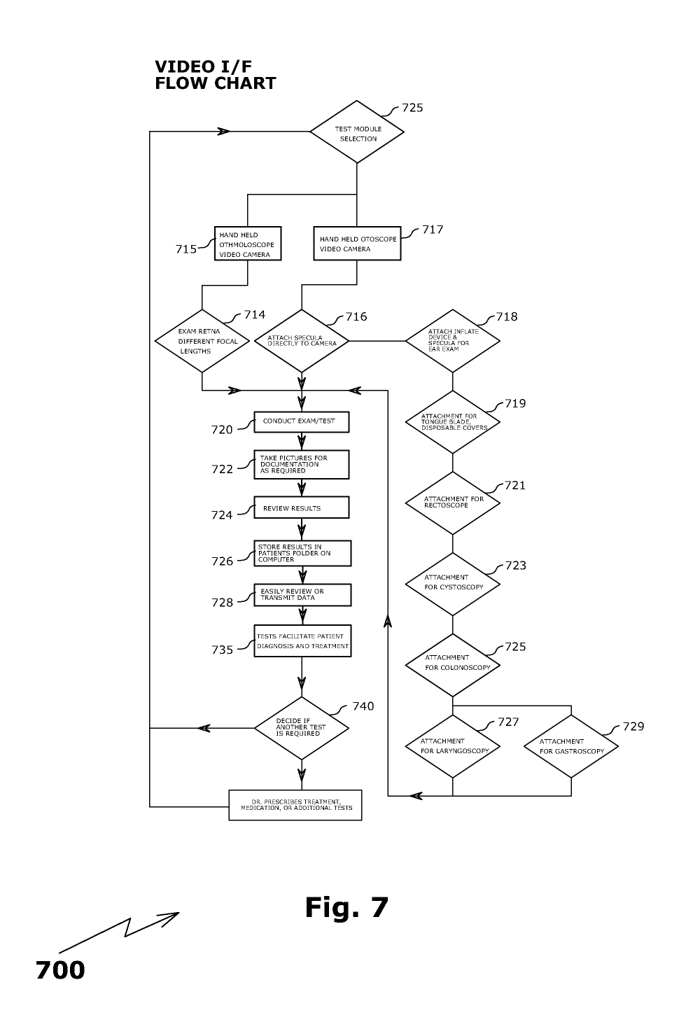

i) FIG. 7 is a video interface flow chart.

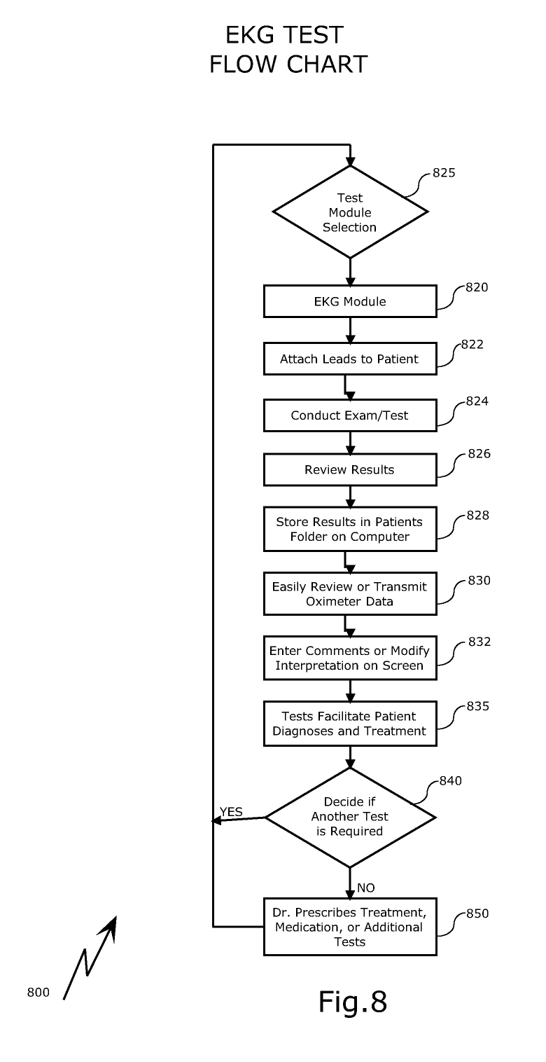

j) FIG. 8 delineates EKG test flow chart.

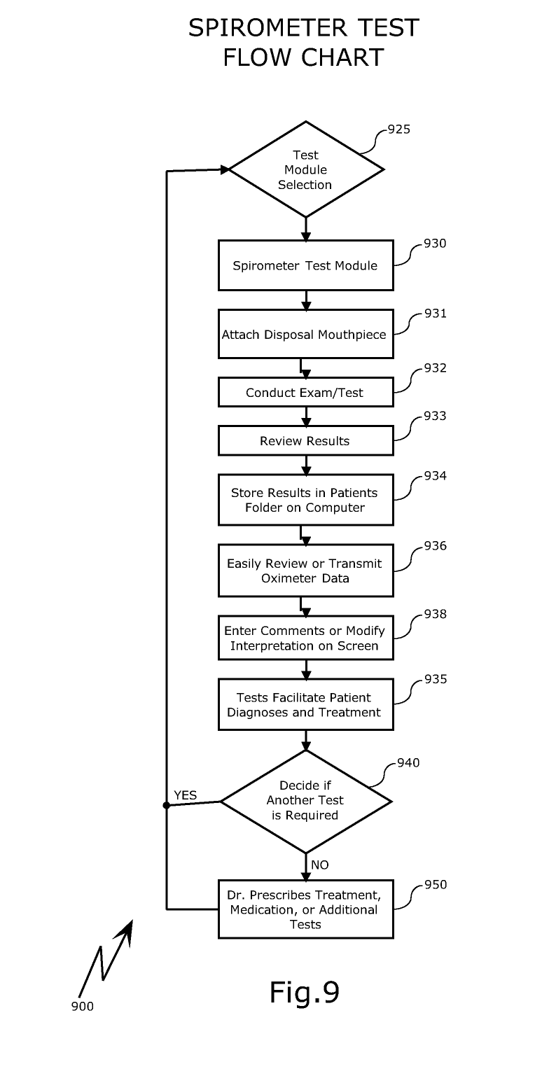

k) FIG. 9 depicts flow of spirometer test.

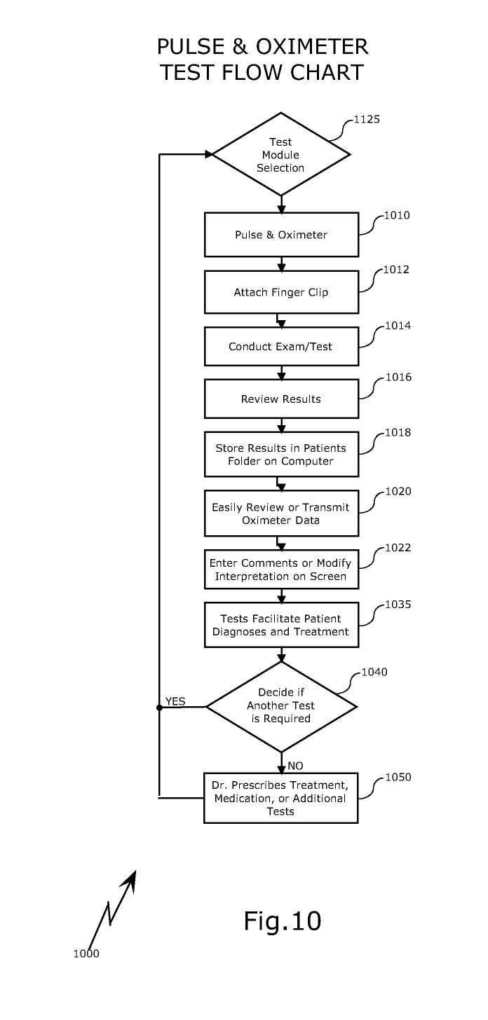

l) FIG. 10 is flow chart of the pulse and oximeter test.

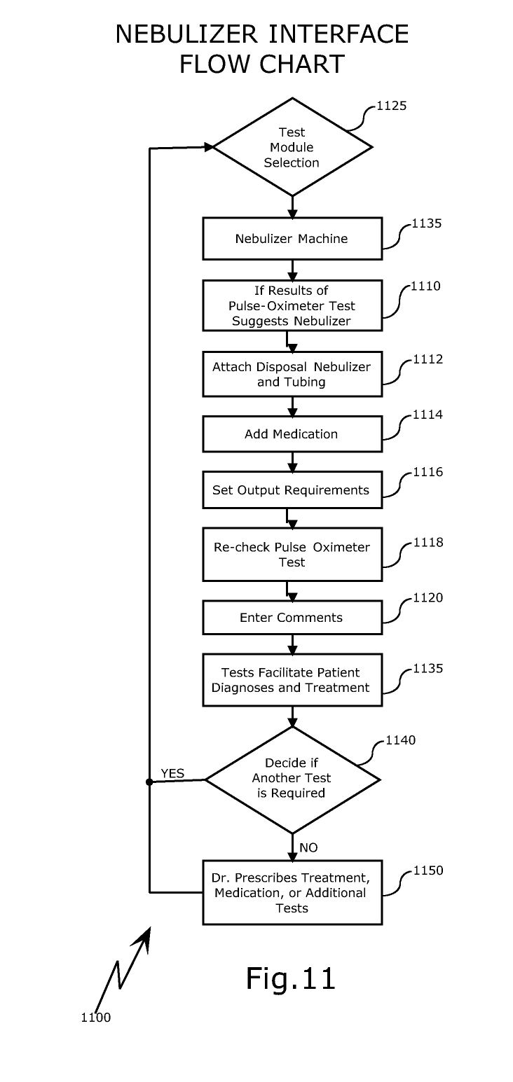

m) FIG. 11 outlines the nebulizer interface flow chart.

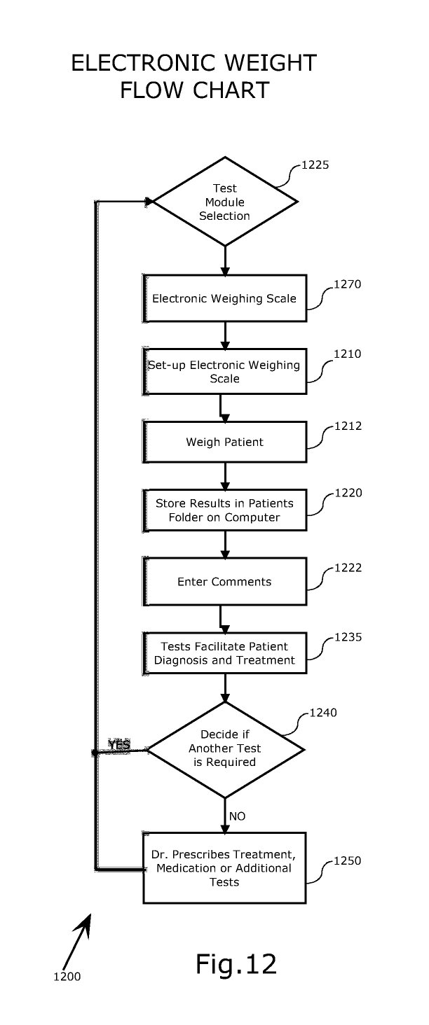

n) FIG. 12 is a rendition of an electronic weight flow chart diagram.

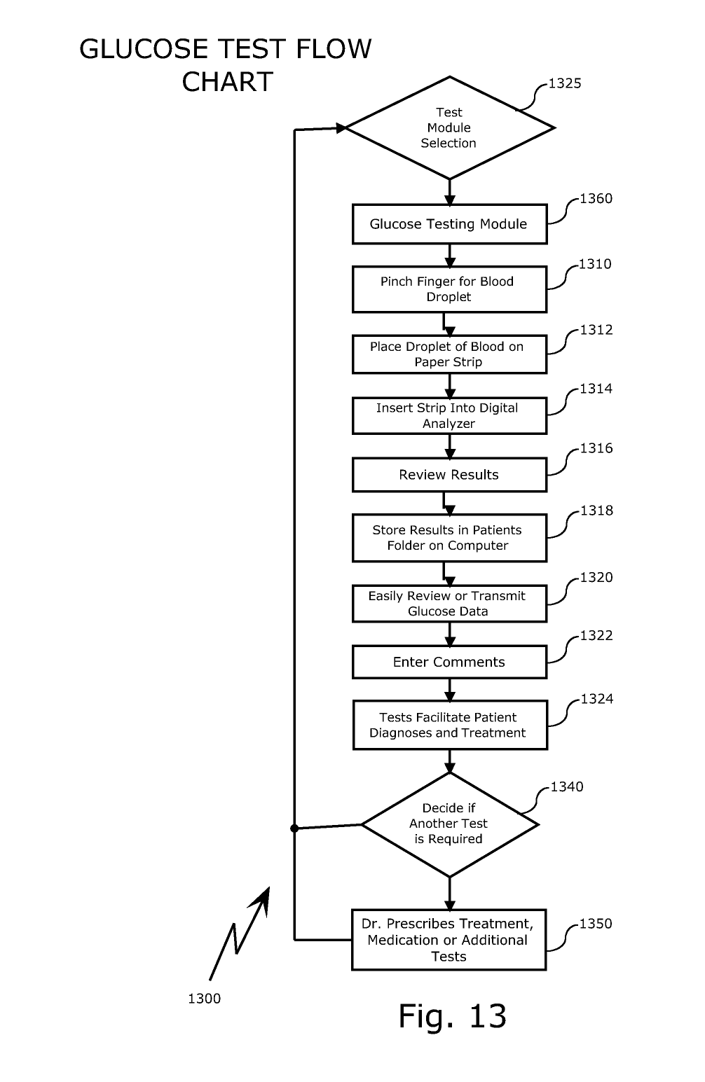

o) FIG. 13 is a glucose test flow chart for diabetic patients.

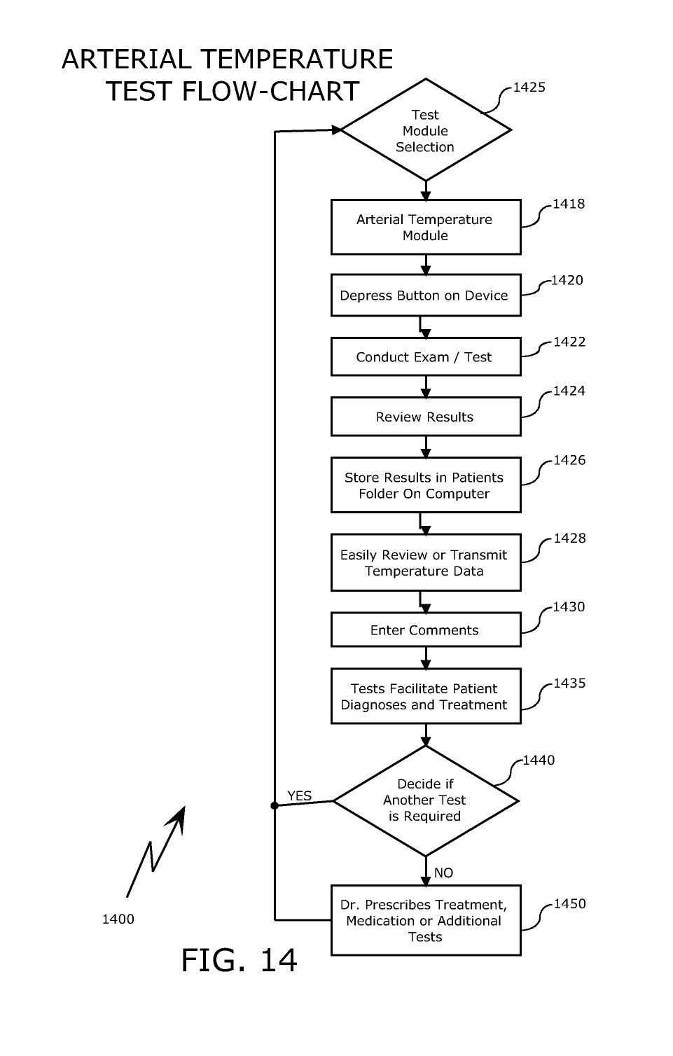

p) FIG. 14 is an arterial temperature test flow chart.

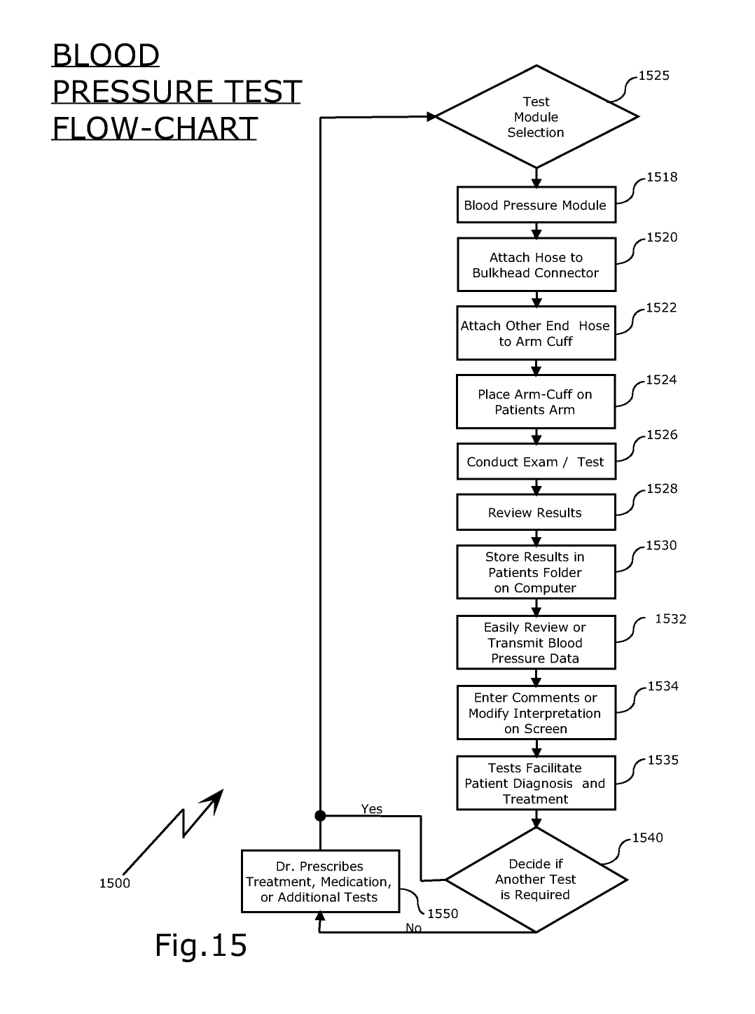

q) FIG. 15 shows blood pressure test data and information flow chart.

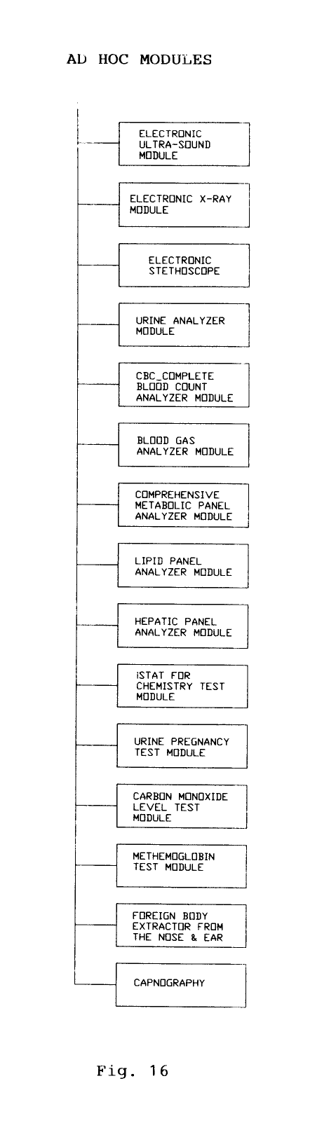

r) FIG. 16 is an enumeration of ad hoc test modules interfaced via USB.

### DETAILED DESCRIPTION OF THE PREFERRED EMBODIMENT

The following is a brief description of the several views of the drawings complete with reference numerals wherein last two least significant digits represent the item (not necessarily tangible) number and the left most one or two digits represent the figure number.

FIG. 1A is a 3D perspective view of the Integrated Portable Medical Diagnostic System suitcase 99 for housing a plurality of modules, convenient carrying handle 98, stability feet 97 and an extendable-retractable handle for rolling and carrying ease. The system is shown complete with computer display 112 and for housing plurality of modules as well as room for expansion and option of adding external modules through a USB port 154 provided for this purpose.

FIG. 1B is a right side view of the Hospital-in-a-Box 100 in partially open/closed position. The display 112 (not visible in this view) is on the underside of the cover of aluminum suitcase 9. The Keyboard 119 is also shown in partially closed position. The dimensions of the Hospital-in-a-Box are such that the keyboard and the display do not interfere with operation of the HIB.

FIG. 1C is side view of the invention in fully closed ready to roll position with retractable handle 101 extended. As is evident from this figure the travel friendly design of the Hospital-in-a-Box includes a telescoping retractable handle 101 which is mounted to the bottom of the aluminum case 99. In the preferred embodiment the inventor utilized wheels directly to the case edge with dimensions such that adjustable stability pads 97 and wheels 96 do not interfere with proper operation of each.

FIG. 2 is a system modules block diagram shown complete with alternating current power line receptacle 202, Direct current power supply charger 204, rechargeable battery pack 205, charger for video camera 206, Switch to Battery 207, switch to alternating current on-site power source 208, Embedded computer internal battery 210 as source of direct current, video camera 216, EKG module 220, PB (Blood Pressure) Module 222, Spirometer 230, Pulse oximeter 240, glucose testing module 260 and arterial thermometer 280 for measuring temperature. Also interfaced to the embedded computer are urine analyzer module 261, CBC complete Blood count analyzer module 262, Blood gas analyzer module 263, comprehensive metabolic panel analyzer module 264, Lipid panel analyzer module 265, and Hepatic panel analyzer module 266.

In addition electronic weighing scale 270, electronic ultrasound module 290, electronic X-Ray module 295 and electronic stethoscope are used on ad hoc basis via the USB interface as they are either bulky or the medical professionals prefer to have the device (such as the stethoscope) on their person for EZ plug in and plug out.

FIG. 3 is another somewhat more detailed rendition of the system modules block diagram of FIG. 2 delineating internal battery and charger 302, power supply 305, charger for video 306, Embedded computer 310 wireless video camera scope 316 and concomitant video receiver 317, arterial temperature module 318, EKG module 320, Blood Pressure module 322, Spirometer 330, Pulse & Oximeter 340, nebulizer 350, Glucose Test Module 360, electronic weighing machine 370, X-Ray module 380 and ultrasound module 390.

FIG. 4 is depicts data system block diagram shown complete with Portable computer 410 wireless video camera scope 416 and concomitant video receiver 417, arterial temperature module 418, EKG module 420, Blood Pressure module 422, Spirometer 430, Pulse & Oximeter 440, nebulizer 450, Glucose Test Module 460, electronic weighing machine 470, X-Ray module 480 and ultrasound module 490.

FIG. 5 shows power sub-system block diagram shown complete with alternating current power 501, alternating current power source 502 (when & where available), rechargeable direct current power pack battery 505, Direct current to alternating current inverter 550, and variety of other direct current sources 590 including vehicle power plug, direct current generator from chemical fuels, and local battery etc.

FIG. 6 is a flow chart of doctor patient interface shown complete with Greet patient 699, discussion of problems and symptoms 602 of the patient directly or vicariously, starting the sonnet system 604, patient information data entry 606, Doctor decides tests deemed necessary 610, Physician decides 620, if the patient should see the results concurrently in real time on the screen for the circumstances and exigencies of the subject patient, if YES face screen towards the patient 622, if NO face screen away from the patient 624 and selection 625 of the modules identified supra and initiation of the main program.

FIG. 7 is a video interface flow chart shown complete with test module selection 725, video camera scope 717, decision block 716 to ascertain whether the specula is attached directly to camera, decision block to attach 718 inflate device and specula for ear examination, conduct examination and tests 720, take pictures 722 for documentation as deemed appropriate, review results 724 with or without patient, store results in patient's folder 726, in the computer, review and transmit oximeter data 728, interpretation of tests 735 to determine patient diagnosis and treatment, decision block 740 to determine if additional tests are warranted, if YES go back to test module selection 725 and if NO prescription/script 750 for treatment, medication and additional tests as appropriate in the professional judgment of the physician in-charge as well as sharing the information with the patient and primary physician if the case so admits.

FIG. 8 delineates EKG test flow chart shown complete with decision block 825 for test module selection, EKG module 820, attaching patches with leads 822 to patient, conducting examination and tests 824, reviewing results 826, storing results 828, in the patient's folder in computer, review and transmission of oximeter data 830, entering comments and/or modifying interpretation 832 on screen, interpretation of tests 835 by medical professionals for diagnosis and treatment, decision block 840 to determine if additional test(s) is/are required, if YES go back to test module selection 825 supra and if NO proceed to or prescription of treatment, medication scripts and additional tests if deemed appropriate.

FIG. 9 depicts flow of spirometer test shown complete with test module selection 925, spirometer test module 930, examination and testing 932, results review 933, storage of results 934 in patients folder in computer, review or transmission of oximeter data 936, Interpretation and commentary 938, patient diagnosis and treatment 935, decision point 940 for need for additional tests, if YES go back to test module selection block 925, if NO treatment, medication and additional tests 950.

FIG. 10 is flow chart of the pulse and oximeter test shown complete with test module selection 1025, pulse and oximeter module 1010, finger clip attachment 1012, examination and testing 1014, results review 1016, storage of results 1018 in patients folder in computer, review or transmission of oximeter data 1020, Interpretation and commentary 1022, patient diagnosis and treatment 1035, decision point 1040 for need for additional tests, if YES go back to test module selection block 1025, if NO treatment, medication and additional tests 1050.

FIG. 11 outlines the nebulizer interface flow chart shown here complete with test module selection 1125, nebulizer machine 1135, verify 1110 pulse oximeter test suggests attachment 1112 of disposal nebulizer and tubing, addition of medication 1114, setting output requirements 1116, Recheck pulse oximeter test 1118, Interpretation and commentary 1120, patient diagnosis and treatment 1135, decision point 1140 for need for additional tests, if YES go back to test module selection block 1125, if NO treatment, medication and additional tests 1150.

FIG. 12 is a rendition of an electronic weight flow chart diagram again shown here complete with test module selection 1225, electronic weighing scales 1270, set up 1210 and initialization of scales 1270, weigh patient 1212, storage of results 1220 in patients folder in computer, Interpretation and commentary 1222, patient diagnosis and treatment 1235, decision point 1240 for need for additional tests, if YES go back to test module selection block 1225, if NO treatment, medication and additional tests 1250.

FIG. 13 is an glucose test flow chart for diabetic patients shown complete with test module selection 1325, glucose testing module 1360, pinch finger for blood droplet 1310, place droplet of blood on paper strip 1312, insert strip into digital analyzer 1314, review results 1316, storage of results 1318 in patients folder in computer, review and transmission of Glucose data Interpretation 1320, add commentary 1322, patient diagnosis and treatment 1324, decision point 1340 for need for additional tests, if YES go back to test module selection block 1325, if NO treatment, medication and additional tests 1350.

FIG. 14 is an arterial temperature test flow chart shown complete with test module selection 1425, Arterial temperature module 1418, initiate test by depressing button on device 1420, conduct examination and test 1422, review of results 1424, storage of results 1426 review and/or transmit temperature data 1428, in patients folder in computer, Interpretation and commentary 1430, patient diagnosis and treatment 1435, decision point 1440 for need for additional tests, if YES go back to test module selection block 1425, if NO treatment, medication and additional tests 1450.

FIG. 15 shows blood pressure test data and information flow chart shown complete with test module selection 1525, Blood Pressure Module 1518, attach hose to Bulkhead connector 1520, attach other end of hose to arm cutoff 1522, position arm cuff on patient's arm, 1524 conduct examination and test 1526, review of results 1528, storage of results 1530 review and/or transmit temperature data 1532, in patients folder in computer, Interpretation and commentary 1534, patient diagnosis and treatment 1535, decision point 1540 for need for additional tests, if YES go back to test module selection block 1525, if NO treatment, medication and additional tests 1550.

FIG. 16 is an enumeration of supplemental test modules to be used on ad hoc need basis generally but not necessarily via Universal Serial Bus portal. The inventor's intention here is to incorporate standard existing medical device modules with accompanying software so that no one but OEM (Original Equipment Manufacturer of the module would have the responsibility, burden and liability of developing the medical device modules and associated operating software. The software has tested and approved algorithms that interpret the results. For example: many years of work have gone into the EKG system and the software that interprets the signals received from the sensors. We want to use each individual module and document the results into our database.

An embedded computer stores all the required software for each module, displays the acquired patient results for physician review and stores these results into the patient's folder on the hard drive. Numerous patients can be evaluated and their medical histories, diagnostic and examination results can be stored on the computer's hard drive until it is convenient to upload the data to a central computer to synchronize all the test and patient information between this portable Medical Diagnostic System, (IPMDS) and the control computer. As an option, the data can be transmitted via a satellite modem to transfer the test data wireless to a central computer and/or EMS service for medical support. The system can operate on AC power or battery power for remote locations.

The main operating system software program can also be used as a stand-alone program to help the physician in making diagnostic decisions on patients. As the physician types in appropriate keywords the program leads him or her into a support menu describing symptoms in detail to support the findings. The program can also cross reference similar symptoms to help determine the patient's condition and list recommended practices, possible additional testing and medications.

Typically the use of the system for each patient comprises at least one test from a group consisting of:

a) Electronic Digital Weighing Machine,

b) Temperature using arterial thermometer,

c) Blood Pressure,

d) Glucose test module,

e) EKG,

f) Electronic X-Ray Module,

g) Electronic Ultrasound Module,

h) Nebulizer Machine Module,

i) Pulse Oximeter,

j) Spirometer,

k) Camera & Video-Scope,

l) a glucose Module, and

m) an ultrasound module,

n) Electronic Stethoscope,

o) Urine Analyzer Module including Urine Pregnancy Test Module,

p) CBC Complete Blood Count Analyzer,

q) iStat for Blood Gas Analyzer Module,

r) Comprehensive Metabolic Panel Analyzer Module,

s) Lipid Panel Analyzer Module,

t) Hepatic Panel Analyzer Module,

u) iStat for Chemistry Test Module,

v) Carbon Monoxide Level Test Module,

w) Methemoglobin Test Module,

x) Ear Nose and Throat Foreign Body Extractor Module,

y) Capnography Module, and

z) A hand held device suitable for and use as
(i) Disposable cover Tongue depressor,
(ii) Colonoscopy,
(iii) Rectoscopy,
(iv) Cystoscopy,
(v) Laryngoscopy; and
(vi) Gastroscopy.
The following are some of the special features of the preferred embodiment contemplated by the inventor.

I. Video camera scope Features:
a) Specula attaches for ear, throat & noise examinations
b) Capable of applying light pressure to the eardrum via special adapter, specula and inflation bulb
c) Wired & wireless versions with an LCD display
d) Video camera has the capability of real time video streaming & snapshots.
e) Modified camera to vary the LED brightness, designed circuit & installed potentiometer on handle for thumb control.
f) Modify the video camera scope by mounting a LCD display to it in order for the physician to get real time feedback via this display as to the location of the specula thereby reducing the possibility of injury during an examination.
g) Two versions for the video camera are version 1 which is hard-wired and version 2, which has a wireless transmitter and receiver. Both versions are capable of displaying images on both the computer and LED scope display concurrently.
h) Video camera scope has an LCD display mounted to it for ease use
i) Video camera scope is capable of real time video streaming & single frame capture of digital images.
j) Video camera scope has LED brightness control knob on handle
k) Video camera scope has focus adjustment knob on handle
l) Video camera scope has the ability to mount special attachment to slightly pressurize the eardrum through the specula via a hand bulb
m) Pictures may be taken for future reference that will stay in the patient's folder
n) Documented pictures can make it easier for the physician to determine if the patient's condition is getting worse
o) Immediate feedback to the patient, they can view exactly what the physician is seeing on the computer screen real time, allowing for the patient to better understand their condition.

II. EKG module Features:
a) Portable 12-channel electrocardiograph
b) Simultaneous real-time waveform viewing
c) Performs quick tests or standard tests
d) Optional narrative interpretation
e) Easily review, transmit and/or export EKG data
f) Enter comments, modify and/or confirm interpretation on screen

III. Spirometer module Features:

a) Portable Spirometer

b) Easy to use data collection

c) Real-time Flow/Volume or Volume/Time traces

d) Disposable mouthpieces

IV. Pulse & Oximeter Module Features:
a) Reads & displays SpO2
b) Reads & displays pulse rate
c) Finger clip

V. Blood Pressure Module Features:

a) Oscillometric measurements for adult & neonatal applications

b) Systolic pressure

c) Diastolic pressure

d) Mean arterial pressure

e) Pulse rate

f) Automatic cuff inflation

g) Automatic cuff overpressure detection

h) Supplied with motion insensitive software

VI. Arterial Temperature Monitor Module Features:

a) Noninvasive temperature measurements

b) Measures arterial blood temperature

VII. Glucose Testing Module Features:
a) To detect if someone is in a diabetic shock
b) Disposable test strips
c) Disposable needles

VIII. Electronic x-ray module Features:
a) For the mouth, teeth, etc.
b) Electronic image displayed on screen.
c) Very sensitive sensor
d) If technology is available we can integrate this option into the HOSPITAL-IN-A-BOX system.

IX. Electronic Ultra-Sound Module Features:
a) Electronic image displayed on screen.
b) If technology is available we can integrate this option into the HOSPITAL-IN-A-BOX system.

X. Features & Uses of the Hand held Module include:
(i) Colonoscopy,
(ii) Rectoscopy,
(iii) Cystoscopy,
(iv) Laryngoscopy,
(v) Gastroscopy; and as
(vi) Disposable cover tongue depressor,

Similarly other modules of past, present or potential use have unique list of features which may be modified as deemed appropriate. Even simple modules like weighing scales and nebulizer module have unique features; notwithstanding this has not been an exhaustive enumeration of features.

### Assembly

1. Purchase and integrate the following OEM medical modules & equipment into a portable carrying case:

a) Electronic Digital Weighing Machine

b) Temperature using arterial thermometer

c) Blood Pressure

d) Glucose test module

e) EKG (Electrocardiogram or electrocardiograph)

f) Electronic X-Ray Module

g) Electronic Ultrasound Module,

h) Nebulizer Machine Module

i) Pulse Oximeter

j) Spirometer and

k) Camera & Video-Scope

Each module is securely mounted and secured within the case or on a sub-frame at its specific location. All the modules are completely wired within the case. Nothing needs to be done. It is ready to use out of the box. Attachments are connected as required. For example: to use the blood pressure module, attach the hose to the bulkhead connector supplied within the case and then attach the arm cuff to the hose. Attach to the arm and start the test. The cuff automatically inflates, takes data, deflates and displays the results.
2. Following additional equipment may be mounted within the case of the Hospital-in-a-Box.

a) Hub

b) Wireless receiver

c) Power supplies

d) Rechargeable battery pack

e) Recharger for video camera scope

f) Electrical receptacle for AC power cord

g) Storage for EKG leads & disposable conductive self-adhesive patches

h) Storage for Spirometer disposable tubes

i) Storage for disposable specula's

j) Storage for disposable latex gloves

k) Storage for blood pressure cuff & hose

It should be noted that for ease of use the inventor has (i) minimized & optimized all wiring and dressed to reduce space requirements. (ii) The system can operate on line while also charging the computer internal battery and video camera internal battery pack and (iii) the case and/or frame structure is organized and may have either a compartment and/or dedicated space for each module.
3. Wire-up and connect each module as required
4. Install computer sub-frame or shelf
5. Install compartment dividers
6. Install computer securely into case or sub-frame
7. Install software programs into the computer for each module
8. Assign communication ports for each module to handshake and operate
9. Set-up Icon's and shortcuts for ease of use
10. Install disposable items in storage areas or a separate case may be utilized for disposals

### Alternate Embodiments

At this time the inventor does not anticipate alternate embodiments beyond the scope of this disclosure and the variation enumerated under the sub-heading of “INVENTOR CONTEMPLATED VARIATIONS” infra but reserves the right to add alternate embodiments when the non-provisional utility application is filed. The inventor applicant also reserves the right to replace any subsystem or module with equivalent functionality for example inventor may replace the personal computer with a microprocessor or equivalent discreet logic function electronic circuits.

### Use & Operation

The operation and use of this device is simple and even intuitive. The device operates as an Integrated Portable Medical Diagnostic System, (IPMD Also known by its three trademark names HOSPITAL-IN-A-BOX, RKRuskat (Stylized) and SCOMET. While the use is obvious and intuitive, the inventor recommends the following sequence of steps.
1. Open Hospital-in-a-Box case
2. Decide if you want line power or battery power
3. Put power switch to the appropriate position
4. If line power is selected, connect power cord to the receptacle on the back of the case.

As an option, a portable self-contained rechargeable battery system mounted within a separate carrying case and may include a suitable Dc to Ac inverter to supply AC power. This system can be plugged into the receptacle on the HIB (Hospital-in-a-Box) case.

This rechargeable system can be recharged from the AC line or from a standard vehicle 12-volt cigarette lighter/power socket. The vehicle power source can recharge the battery pack while also supplying power to the HOSPITAL-IN-A-BOX system. As a further option the HOSPITAL-IN-A-BOX system can be powered via an extension wire from the rechargeable system while the rechargeable system is located within the vehicle and plugged into the vehicle 12-volt cigarette lighter/power socket producing unlimited operation as long as the vehicle engine is running.

If battery power is selected, connect the self-contained portable rechargeable battery and inverter system to the HOSPITAL-IN-A-BOX system receptacle with the appropriate power cord.

As another option, the HOSPITAL-IN-A-BOX system can contain the rechargeable batteries internally and may also contain the inverter, which will allow the system to be automatically operated on battery power.
5. Start up computer and wait for it to completely boot up.
6. Choose the medical device required for the patient test & remove the sensor and any disposal attachments that might be required from the case.
7. Start the main program by clicking on the appropriate Icon on the computer screen
8. When the program starts, follow directions to perform the test on the patient
9. At end of patient test, save results to the hard-drive in the patient's folder, add comments as required in appropriate areas.
10. Use video camera scope to view internal ear, nose, eye, etc. and also document the patient's condition, such as contusions, etc. and save digital images (snapshots) to the patient's folder.
   The following examples delineate special customized instructions for the modules discussed infra.

I. Oximeter Measurement

1. Select Oximeter Icon

2. Select OK at message “this version is for evaluation only”

3. Open Comm. port

4. Put on finger sensor

5. Select Comm. Port #1

6. Select OK.

7. Read and watch readings.

8. When satisfied:

9. File, (upper left hand corner)

10. Begin capture, (F12)

11. Save data to C: Data

12. Data starts capturing

13. Save data

14. Close Communication port

15. Exit program.

II. SPIROMETER MEASUREMENT

1. Select Spirometer Icon

2. Select OK to save settings reminder

3. Select Data, upper tool bar

4. Add New Patient

5. Fill out form

6. Highlight patients name

7. Select new examination

8. Comment on medication if required

9. Message does not apply, select OK

10. Blow into instrument twice

11. Accept/Done or Reject

12. Select File

13. Save As

14. Enter name

15. Save data

III. EKG:

1. Select EKG Icon.

2. Choose patients name or

3. File

4. New

5. Fill out patient form

6. Highlight patients name

7. Select EKG Icon from toolbar

8. Run EKG

9. Save data as required

IV. Video Camera/Scope
1. Select camera Icon
2. Turn camera on by using the upper switch. Hold switch down until an image appears on the LCD display
3. Full screen, Double click left mouse
4. To reduce screen, Double click left mouse again
5. To take picture, momentarily press the upper switch down
6. Camera stacks up to ten pictures internally
7. To download picture into the computer
   a) Use the lower switch to scroll through the pictures one at a time by pushing the lower switch up.
   b) When the desired picture is on the LCD display use the mouse to click the Camera Icon on the wood frame
   c) Select Save
   d) Select Path, (my documents, my pictures)
   e) Enter File name
   f) Save
   g) Close the Save window
   h) To exit camera mode, select yellow arrow, (lower right side of wood frame)
   i) To return camera to real time, momentarily press upper switch down
   j) To save additional pictures go back to step 8 and repeat sequence as required.

V. Temperature Measurement
1. With probe flush on the center of the forehead, depress button and keep this button depressed.
2. Slowly slide probe across the forehead into the hairline.
3. Place probe on head behind the earlobe.
4. Release the button when the LED temperature numbers stabilize.
5. Remove probe from the head.
6. A document patient temperature reading manually until software program is developed.

VI. Blood Pressure Metric
1. Select Blood pressure Icon on screen
2. Connect hose to cuff and install cuff on arm
3. Insert hose into bulkhead connector
4. Start BP measurement.
5. When Reply is shown as below, (Right side of screen)
6. Reply O
7. Reply K
8. Select Get BP data
9. Select Read data
10. Read and analyze data
11. Document patient blood pressure readings manually until software program are developed.

### Inventor Contemplated Variations

The basic concept of Integrated Portable Medical Diagnostic System disclosed here would be difficult to design around. The inventor has given a non-limiting description of this invention. Due to the simplicity and elegance of the design of this invention designing around it is very difficult if not impossible. The concept of Hospital-in-a-Box System disclosed here would be difficult to design around. Nonetheless many changes may be made to this design without deviating from the spirit of this invention. Examples of such contemplated variations include the following:
1. The value and the tolerance of various electronic components may be modified.
2. The suitcase box may be built with newer materials, technologies and processes as they become available.
3. The PCB and its conductors can be made of different materials as they become available due to the technological progress in polymer chemistry
4. Design a small dedicated computer to replace the laptop
5. Design a computer program to operate the complete system as mentioned above on a handheld palm type computer. This software would probably have to be compatible with Windows CE and then software and drivers would need to be written for all the medical device modules that operate on Windows.
6. Decrease the overall size of the case and/or modules
7. Integrate different modules with different functions
8. Design a computer motherboard to support all the functions of the various modules and develop the required software within one or multiple operating programs
9. The system may be upscaled or downscaled in size, weight, ease of use, cost, etc.
10. Design a solar powered rechargeable battery system that is integrated into the HOSPITAL-IN-A-BOX system.
11. The value and the tolerance of various electronic components may be modified.
12. The system or any part of it may be built with newer materials, technologies and processes as they become available.
13. The number and types of modules and tests may be varied.
14. The PCB and its conductors can be made of different materials as they become available due to the technological progress in polymer chemistry
15. Additional complimentary and complementary functions and features may be added.
16. A more economical or an upscale version of the device may be adapted.
17. Numerous medical instrument modules are packaged and integrated into a single portable carrying case or a knapsack can be used for military and remote location use.
18. Portable HOSPITAL-IN-A-BOX system operates from the AC line or, from the portable rechargeable battery pack integrated with a Dc to Ac inverter or, from a vehicle 12-volt cigarette lighter/power socket powering the portable rechargeable battery pack and inverter system.
19. Satellite modem to transfer test data wireless to central computer/EMS service.
20. Video camera scope has an LCD display mounted to it for ease use
21. Video camera scope is capable of real time video streaming & single frame capture of digital images.
22. Video camera scope has LED brightness control knob on handle
23. Video camera scope has focus adjustment knob on handle
24. Video camera scope has the ability to mount special attachment to slightly pressurize the eardrum through the specula via a hand bulb
25. Pictures may be taken for future reference that will stay in the patient's folder as documented pictures can make it easier for the physician to determine if the patient's condition is getting worse
26. Immediate feedback to the patient, they can view exactly what the physician is seeing on the computer screen real time, allowing for the patient to better understand their condition.
27. Additional complimentary and complementary functions and features may be added.
28. A more economical or an upscale version of the device may be adapted.
29. Other modules as they become available may be interfaced to this system via the USB port
30. Other changes such as aesthetics and substitution of newer materials as they become available, which substantially perform the same function in substantially the same manner with substantially the same result without deviating from the spirit of the invention may be made.

### REFERENCE NUMBER CONCORDANCE

Following is a listing of the components uses in this embodiment arranged in ascending order of the reference numerals for ready reference of the reader.
97=HIB Stability Pads
98=Convenient carrying handle
99=Integrated Portable Medical Diagnostic System suitcase
100=HIB Embodiment of FIG. 1 generally
101=Telescopic Pull-out & Retractable handle for rolling HIB
112=Display
119=Fold over keyboard
152=Power Receptacle Socket
154=USB port
200=Embodiment of FIG. 2 generally
202=Alternating current power line receptacle
204=Direct current power supply charger
205=Rechargeable battery pack
206=Charger for video camera
207=Switch to Battery
208=Switch to alternating current on-site power source
210=Portable computer internal battery as source of direct current
216=Video camera & Scope
220=EKG module
222=PB (Blood Pressure Meter)
230=Spirometer
240=Pulse oximeter
260=Glucose Test Module
261=Urine analyzer module including urine pregnancy test
262=CBC complete Blood count analyzer module
263=Blood gas analyzer module
264=Comprehensive metabolic panel analyzer module
265=Lipid panel analyzer module
266=Hepatic panel analyzer module
270=Electronic weighing scale
280=Arterial thermometer for measuring temperature.
290=Electronic Ultrasound Module
295=Electronic X-Ray Module
296=Electronic Stethoscope
300=Embodiments of FIG. 3 generally
302=Internal battery and charger 30
305=Power supply
306=Charger for video
310=Portable computer
316=wireless video camera scope
317=Video receiver
318=Arterial temperature module
320=EKG module
322=Blood Pressure module
330=Spirometer
340=Pulse & Oximeter
350=Nebulizer Machine Module
360=Glucose Test Module
370=Electronic weighing machine
380=X-Ray module
390=Ultrasound module
400=Embodiment of FIG. 4 generally
410=Portable computer
416=Wireless video camera scope
417=Video receiver
418=Arterial temperature module
420=EKG module 420
422=Blood Pressure module
430=Spirometer 430
440=Pulse & Oximeter
450=Nebulizer Module
460=Glucose Test Module
470=Electronic weighing machine
480=X-Ray module
490=Ultrasound Module 490.
500=Embodiment of FIG. 5 generally
501=Alternating current power
502=Alternating current power source (if when & where available)
505=Rechargeable direct current power pack battery
550=Direct current to alternating current inverter
590=Variety of other direct current sources including vehicle power plug, direct current generator from chemical fuels & local battery etc.
600=Embodiment of FIG. 6 generally
602=Discussion of problems and symptoms of the patient directly or vicariously
604=Starting the sonnet system
606=Patient information data entry
610=Doctor decides tests deemed necessary for each patient on a case by case basis.
620=Physician decides if whether and when the patient should see the results concurrently in real time on the screen for the circumstances and exigencies of the subject patient, if YES face screen towards the patient 622, if NO face screen away from the patient 624 and selection 625 of the modules identified supra and initiation of the main program.
699=Greet Patient
700=Embodiment of FIG. 7 generally
716=Video camera scope decision block
717=Verification to ascertain whether the specula is attached directly to camera
718=Decision block to Inflate device and specula for ear examination
720=Conduct examination and tests
722=Take pictures for documentation as deemed appropriate
724=Review Results with or without patient
726=Store results in patient's folder in the computer
728=Review and transmit oximeter data
735=Interpretation of tests to determine patient diagnosis and treatment
740=Decision block to determine if additional tests are warranted
750=Prescription/script for treatment, medication and additional tests
800=Embodiment of FIG. 8 generally
825=Decision block for test module selection
820=EKG module
822=Attaching patches with leads to patient
824=Conducting examination and tests
826=Reviewing results
828=Storing results in the patient's folder in computer
830=Review and transmission of oximeter data
832=Entering comments and/or modifying interpretation on screen display screen
835=Interpretation of tests by medical professionals for diagnosis and treatment
840=Decision block to determine if additional test(s) is/are required or deemed appropriate
900=Embodiment of FIG. 9 generally
925=Test module selection
930=Spirometer test module
932=Examination and testing
933=Review of results
934=Storage of results in patients folder in the computer
936=Review and/or transmission of oximeter data
938=Interpretation and commentary
935=Patient diagnosis and treatment
940=Decision point to ascertain need for additional tests
950=Treatment, medication and additional tests.
1000=Embodiment of FIG. 10 generally
1010=Pulse and oximeter module
1012=Attachment of Finger clip
1014=Examination and testing
1016=Results review
1018=Storage of results in patients folder in the patient computer
1020=Review or transmission of oximeter data
1022=Interpretation and commentary
1025=Test module selection decision block
1035=Patient diagnosis and treatment
1040=Decision point for need for additional tests
1050=Treatment, medication and additional tests
1100=Embodiment of FIG. 11 generally
1110=Verification that pulse oximeter tests suggest nebulizer
1112=Attachment of disposal nebulizer and tubing
1114=Addition of medication
1116=Setting output requirements
1118=Recheck pulse oximeter test
1120=Interpretation and commentary
1125=Test module selection decision block
1135=Patient diagnosis and treatment on Nebulizer machine module
1140=Decision point to ascertain need for additional tests
1150=Treatment, medication and additional tests
1200=Embodiment of FIG. 12 generally
1210=Set up and initialization of scales
1212=Weigh patient
1220=Storage of results in patients folder in the patient computer
1222=Interpretation and commentary
1225=Test module selection decision block
1270=Electronic weighing scales
1235=Patient diagnosis and treatment
1240=Decision point to ascertain need for additional tests
1250=Treatment, medication and additional tests
1300=Embodiment of FIG. 13 generally
1312=Pinch finger for blood droplet
1312=Place droplet of blood on paper strip
1314=Insert strip into digital analyzer
1316=Review results
1318=Storage of results in patients folder in the patient computer
1320=Review and/or transmission of Glucose data
1322=Add commentary
1324=Patient diagnosis and treatment
1340=Decision point to ascertain need for additional tests
1350=Treatment, medication and additional tests
1360=Glucose testing module
1400=Embodiment of FIG. 14 generally
1418=Arterial temperature module
1420=Initiate test by depressing button on device
1422=Conduct examination and test
1424=Review of results
1425=Test module selection decision point 1
1426=Storage of results
1428=Review and/or transmit temperature data
1430=Interpretation and commentary
1435=patient diagnosis and treatment
1440=Decision point to ascertain need for additional tests
1450=Treatment, medication and additional tests
1500=Embodiment of FIG. 15 generally
1525=Test module selection decision point
1518=Blood Pressure Module
1520=Attach hose to Bulkhead connector
1522=Attach other end of hose to arm cutoff
1524=Position arm cuff on patient's arm
1526=Conduct examination and tests
1528=Review of results
1530=Storage of results
1532=Review and/or transmit temperature data in patient electronic folder
1534=Interpretation and commentary
1535=Patient diagnosis and treatment
1540=Decision point for need for additional tests.
1550=Treatment, Medication and Additional Tests

### DEFINITIONS AND ACRONYMS

A great care has been taken to use words with their conventional dictionary definitions. Following definitions are included here for clarification.
3D=Three Dimensional
AC=Alternating Current Power
AD Hoc=Test modules to be added on need basis
BP=Blood pressure Metric
CPU=Central Processing Unit
DC=Direct Current Power
DIY=Do It Yourself
EKG=Electrocardiogram
HIB=Hospital-in-a-Box
IC=Integrated Circuit
I/O=Input and Output
Integrated=Combination of two entities to act like one
Interface=Junction between two dissimilar entities
LCD=Liquid Crystal Display
LED=Light Emitting Diode
OEM=Original Equipment Manufacturer
PB=Blood Pressure Module
PCB=Printed Circuit Board
Pixel=Smallest/finest resolution on a display.
Symmetrical=The shape of an object of integrated entity which can be divided into two along some axis through the object or the integrated entity such that the two halves form mirror image of each other.
USB=Universal Serial Bus Port or Portal

While this invention has been described with reference to illustrative embodiments, this description is not intended to be construed in a limiting sense. Various modifications and combinations of the illustrative embodiments as well as other embodiments of the invention will be apparent to a person of average skill in the art upon reference to this description. It is therefore contemplated that the appended claim(s) cover any such modifications, embodiments as fall within the true scope of this invention.

The inventor claims:

1. An Integrated Portable Medical Diagnostic System comprising;
a) A roll able self-contained suitcase;
b) a central processing unit as an embedded computer housed in said suitcase;
c) a display connected to said central processing unit;
d) plurality of storage areas connected to said central processing unit;
e) a power module for supplying power needs of said portable medical diagnostic system;
f) a video scope connected to said central processing unit;
g) an EKG module interfaced to said central processing unit;
h) a blood pressure monitor module interfaced to said central processing unit;
i) a spirometer module interfaced to said central processing unit;
j) a pulse and oximeter interfaced to said central processing unit; and
k) an arterial thermometer interfaced to said central processing unit.

2. The Integrated Portable Medical Diagnostic System of claim 1 wherein said suitcase includes a retractable pull out handle and a convenient fixed carrying handle.

3. The Integrated Portable Medical Diagnostic System of claim 1 wherein said suitcase includes a power sub-system and a camera & video scope housed in said suitcase and interfaced to said central processing unit.

4. The Integrated Portable Medical Diagnostic System of claim 3 wherein said power sub-system comprises a power module with alternating current power means and rechargeable direct current power battery pack and a generator for emergency power backup.

5. An Integrated Portable Medical Diagnostic System comprising;
a) a central processing unit as a computer;
b) a display connected to said central processing unit;
c) plurality of storage areas connected to said central processing unit;
d) a video scope connected to said central processing unit; and
at least one medical module from the group consisting of:
(i) an EKG module,
(ii) a blood pressure monitor,
(iii) a spirometer,
(iv) a pulse and oximeter,
(v) an arterial thermometer,
(vi) arterial temperature module,
(vii) a nebulizer
(viii) a glucose Module
(ix) an electronic weighing machine
(x) An X-Ray module
(xi) an ultrasound module; and
e) wherein said medical module is interfaced to said central processing unit.

6. The Integrated Portable Medical Diagnostic System of claim 5 which includes at least one ad hoc medical test module from the group consisting of:
(i) Electronic Ultra Sound Module,
(ii) Electronic X-Ray Module,
(iii) Electronic Stethoscope,
(iv) Urine Analyzer Module,
(v) CBC Complete Blood Count Analyzer,
(vi) iStat for Blood Gas Analyzer Module,
(vii) Comprehensive Metabolic Panel Analyzer Module,
(viii) Lipid Panel Analyzer Module,
(ix) Hepatic Panel Analyzer Module,
(x) iStat for Chemistry Test Module,
(xi) Urine Pregnancy Test Module,
(xii) Carbon Monoxide Level Test Module,
(xiii) Methemoglobin Test Module,
(xiv) Ear Nose and Throat Foreign Body Extractor Module; and
(xv) Capnography Module.

7. The Integrated Portable Medical Diagnostic System of claim 6 wherein said ad hoc module is connected to said system through a Universal Serial Bus port.

8. The Integrated Portable Medical Diagnostic System of claim 5 which includes a hand held device with at least one test selected from a group consisting of:
(i) Colonoscopy,
(ii) Rectoscopy,
(iii) Cystoscopy,
(iv) Gastroscopy,
(v) Laryngoscopy, and as
(vi) Tongue depressor with disposable cover.

9. The process of medical diagnosis and tests comprising the steps of:
a) opening system carrying case;
b) selecting alternating current line power or direct current battery power;
c) starting central processing unit computer and waiting for it to completely boot up;
d) choosing the medical device required for the patient test & remove the sensor and any disposal attachments that might be required from the case;
e) initiating at least one computer program by clicking on the appropriate Icon on the computer screen;
f) performing test protocol on the patient as directed by the instructions from the system;
g) saving test results to the hard-drive at the end of the test in the patient's; and
h) adding comments as required in appropriate areas.

10. The process of medical diagnosis and tests of claim 9 which includes at least one test from a group consisting of;
(i) an EKG module,
(ii) a blood pressure monitor module,
(iii) a spirometer module,
(iv) a pulse and oximeter module,
(v) an arterial thermometer,
(vi) arterial temperature module,
(vii) a nebulizer,
(viii) a glucose Module,
(ix) an electronic weighing machine,
(x) an X-Ray module; and
(xi) an ultrasound module.

11. The process of medical diagnosis and tests of claim 9 which includes at least one ad hoc test from a group consisting of;
(i) Electronic Ultra Sound Module,
(ii) Electronic X-Ray Module,
(iii) Electronic Stethoscope,
(iv) Urine Analyzer Module,
(v) CBC Complete Blood Count Analyzer,
(vi) iStat for Blood Gas Analyzer Module,
(vii) Comprehensive Metabolic Panel Analyzer Module,
(viii) Lipid Panel Analyzer Module,
(ix) Hepatic Panel Analyzer Module,
(x) iStat for Chemistry Test Module,
(xi) Urine Pregnancy Test Module,
(xii) Carbon Monoxide Level Test Module,
(xiii) Methemoglobin Test Module,
(xiv) Ear Nose and Throat Foreign Body Extractor Module; and
(xvi) Capnography Module.

12. The process of medical diagnosis and tests of claim 11 wherein said ad hoc module is connected to said system through a Universal Serial Bus port.

13. The process of medical diagnosis and testing of claim 9 which includes a hand held device with at least one test selected from a group consisting of:
(vii) Colonoscopy,
(viii) Rectoscopy,
(ix) Cystoscopy,
(x) Gastroscopy,
(xi) Laryngoscopy, and as
(xii) Tongue depressor with disposable cover.

14. The process of medical diagnosis and tests of claim 9 which entails use of a video camera scope to view internal ear, nose, eye, etc. and also document the patient's condition, such as contusions, etc. and save digital images (snapshots) to the patient's folder.

15. A process of building a portable medical diagnostic and testing system comprising the steps of:
a) integrating five or more OEM modules from the group consisting of the following into a portable carrying case;
(i) an Electronic Digital Weighing Machine;
(ii) a temperature using arterial thermometer;
(iii) Blood Pressure;
(iv) Glucose test module;
(v) EKG;
(vi) Electronic X-Ray Module;
(vii) Electronic Ultrasound Module;
(viii) Nebulizer Machine Module;
(ix) Pulse Oximeter;
(x) Spirometer; and
(xi) Camera & Video-Scope
b) optionally adding and integrating at least additional equipment from the group consisting of;
(i) hub;
(ii) wireless receiver;
(iii) power supply;
(iv) rechargeable battery pack;
(v) recharger for battery;
(vi) recharger for video camera scope;
(vii) electrical receptacle for AC power cord;
(viii) storage for EKG leads & disposable conductive self-adhesive patches;
(ix) storage for Spirometer disposable tubes;
(x) storage for disposable speculas;
(xi) storage for disposable latex gloves; and
(xii) storage for blood pressure cuff & hose: and
c) connecting each module as directed by the instructions;
d) installing computer sub-frame or shelf;
e) installing compartment dividers;
f) installing computer and all equipment selected for the configuration securely into a suitcase;
g) installing software programs into the computer for each module selected;
h) assigning communication ports for each module for proper hardware software interface protocol; and
i) setting-up Icon's and shortcuts for ease of use.

16. The process of building a portable medical diagnostic and testing system of claim 15 which includes at least one ad hoc medical test module from the group consisting of:
(i) Electronic Ultra Sound Module,
(ii) Electronic X-Ray Module,
(iii) Electronic Stethoscope,
(iv) Urine Analyzer Module,
(v) CBC Complete Blood Count Analyzer,
(vi) iStat for Blood Gas Analyzer Module,
(vii) Comprehensive Metabolic Panel Analyzer Module,
(viii) Lipid Panel Analyzer Module,
(ix) Hepatic Panel Analyzer Module,
(x) iStat for Chemistry Test Module,
(xi) Urine Pregnancy Test Module,
(xii) Carbon Monoxide Level Test Module,
(xiii) Methemoglobin Test Module,
(xiv) Ear Nose and Throat Foreign Body Extractor Module; and
(xvii) Capnography Module.

17. The process of building a portable medical diagnostic and testing system of claim 15 wherein said ad hoc module is connected to said system through a Universal Serial Bus port.

18. The process of building a portable medical diagnostic testing system of claim 15 which includes a hand held device with at least one test selected from a group consisting of:
(xiii) Colonoscopy,
(xiv) Rectoscopy,
(xv) Cystoscopy,
(xvi) Gastroscopy,
(xvii) Laryngoscopy, and as
(xviii) Tongue depressor with disposable cover.
